SEQUENCE LISTING (10 sequences)

SEQ ID NO: 1 - DNA, 45 bp, Artificial sequence
  Primer sequence
ctaatacgactcactatagggcaagcagtggtatcaacgcagagt

SEQ ID NO: 2 - DNA, 21 bp, Artificial sequence
  Primer sequence
ctaatacgactcactataggg

SEQ ID NO: 3 - DNA, 32 bp, Artificial sequence
  Primer sequence
cgcggctagcacactctcccctgttgaagctc

SEQ ID NO: 4 - DNA, 23 bp, Artificial sequence
  Primer sequence
tcatttacccggggacagggaga

SEQ ID NO: 5 - DNA, 16 bp, Artificial sequence
  Primer sequence
gtaaaacgacggccag

SEQ ID NO: 6 - DNA, 17 bp, Artificial sequence
  Primer sequence
caggaaacagctatgac

SEQ ID NO: 7 - DNA, 705 bp, Homo sapiens
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcaactacttagcctggtaccaacagaaacctggccaggctcccaggctcctcatctttgatgcatccaacagggccactggcatcccagccagggtcagaggcagtgggtctgggacagacttcactctcaccatcagcagcctagagcctgaagattttgcagtttattactgtcagcagcgtagcaactggccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - DNA, 1422 bp, Homo sapiens
atggagtttgtgctgagctgggttttccttgttgcaatattaaaaggtgtccagtgtgaggatcagctggtgcagtctgggggaggcttggtacatccggaggggtccctgagactctcctgtgcaggctctggattcaccttcagtaactatgctatgaactgggttcgccaggctccaggaaaaggtctggagtgggtatcaactgttgatactggtggtagcacatactatgcagactccgtgaagggccgattcaccatctccagagacaatgccaagaactccttgtatcttcaaatgaacagcctgagagccgaggacatggctgtgtattactgtgcaagagatcttttggttcggggagccctcggctactactacggtatggacgtctggggccaagggaccacggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 9 - protein, 234 aa, Homo sapiens
MEAPAQLLFLLLLWLPDTTGEIVLTQSPATLSLSPGERATLSCRASQSVSNYLAWYQQKPGQAPRLLIFDASNRATGIPARVRGSGSGTDFTLTISSLEPEDFAVYYCQQRSNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 473 aa, Homo sapiens
MEFVLSWVFLVAILKGVQCEDQLVQSGGGLVHPEGSLRLSCAGSGFTFSNYAMNWVRQAPGKGLEWVSTVDTGGSTYYADSVKGRFTISRDNAKNSLYLQMNSLRAEDMAVYYCARDLLVRGALGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2008/0305516 A1
(43) Pub. Date: Dec. 11, 2008

(54) Method of Producing Antibodies

(76) Inventors: Kristian Kjaergaard, Ballerup (DK); Susan Bang, Bagsvaerd (DK)

Correspondence Address: NOVO NORDISK, INC.;INTELLECTUAL PROPERTY DEPARTMENT, 100 COLLEGE ROAD WEST, PRINCETON, NJ, 08540, US

(21) Appl. No.: 11/720,028

(22) PCT Filed: Nov. 30, 2005
(86) PCT No.: PCT/EP2005/056351

§ 371 Date: Mar. 20, 2008

(30) Foreign Application Priority Data
Nov. 30, 2004 (DK) PA 2004 01871
Feb. 17, 2005 (DK) PA 2005 00246

(51) Int. Cl. C12P 39/00; C12P 21/04; C07K 16/00

(52) U.S. Cl. 435/42; 435/69.6; 530/387.1

(57) ABSTRACT

Methods for producing immunoglobulin molecules or immunologically functional immunoglobulin fragments, the fragments comprising at least functional portions of the variable domains of immunoglobulin heavy and light chains, are described. The methods comprise producing the heavy and the light chains in two separate host cells and refolding the immunoglobulin molecule or fragment ex vivo.

### FIELD OF THE INVENTION

The present invention relates to a method for producing an immunoglobulin molecule or an immunological functional immunoglobulin fragment. In this method, an immunoglobulin light and heavy chain sequences can be expressed in separate host cells in the same cell culture, followed by ex vivo assembly of the immunoglobulin or immunoglobulin fragment.

### BACKGROUND OF THE INVENTION

Monoclonal antibodies can either be produced by hybridoma technology or by recombinant expression. Recombinant expression offers more options in improving antibody design and production. Various formats of antibodies or antibody fragments can be expressed recombinantly, and properties like affinity, specificity and glycosylation can be altered by genetic engineering to improve the characteristics of the antibodies or antibody fragments. The productivity of recombinantly expressed antibodies normally exceed the yield obtained from hybridomas, resulting in lower production costs for recombinantly produced antibodies.

U.S. Pat. No. 6,331,415 (Cabilly et al.) describes a method for the recombinant production of immunoglobulin where the heavy and light chains are expressed simultaneously from a single vector or from two separate vectors in a single cell.

Wibbenmeyer et al., (1999, Biochim Biophys Acta 1430(2):191-202) and Lee and Kwak (2003, J. Biotechnology 101:189-198) both describe the production of monoclonal antibodies from separately produced heavy and light chains, using plasmids expressed in separate cultures of E. coli. The H- and L-chains were expressed as inclusion bodies, purified from cell lysates and refolded in vitro.

U.S. Pat. No. 5,643,745 describes a method for producing a heterodimer by culturing a filaqmentous fungus containing two nuclei, the first of which modified to express the first subunit of the heterodimer, and the second of which modified to express the second subunit.

The one cell expression system is convenient since the antibody will be structurally ready for use after purification. However, other factors might be difficult to control such as, e.g., expressing the light and heavy chains in the desired ratios in order to obtain optimal yields. Expression in gram-negative E. coli, on the other hand, can result in contamination with endotoxins, and expression of full length immunoglobulins can be problematic.

Accordingly, alternative methods for producing recombinant antibodies, tailored to suit specific needs and which overcome the above problems, are still needed.

### SUMMARY OF THE INVENTION

The present invention provides a method for producing an immunoglobulin molecule or an immunologically functional immunoglobulin fragment comprising at least a functional portion of the variable domains of the immunoglobulin heavy and light chains, the method comprising the steps of producing the heavy and the light chains in two separate host cells selected from the group consisting of eukaryotic cells and gram positive bacteria; and refolding the immunoglobulin molecule or immunologically functional immunoglobulin fragment ex vivo.

Accordingly, the invention provides a method of producing an immunoglobulin molecule or an immunologically functional fragment thereof, the method comprising:

(a) transforming a first host cell with a first nucleic acid comprising a nucleotide sequence encoding a first polypeptide comprising at least the variable domain of an immunoglobulin heavy chain;

(b) transforming a second host cell with a second nucleic acid comprising a nucleotide sequence encoding a second polypeptide comprising at least the variable domain of an immunoglobulin light chain;

(c) expressing the first and second nucleic acid sequences;

(d) purifying the first and second polypeptides; and

(e) allowing the first and second polypeptides to refold to form an immunoglobulin molecule or immunologically functional immunoglobulin fragment;

wherein the first and second host cells are separately selected from the group consisting of a eukaryotic cell and a Gram-positive bacterium.

The immunoglobulin molecule can be selected from, for example, an IgA, an IgD, an IgE, an IgG, and an IgM immunoglobulin. The immunologically functional fragment can be selected from, for example, a Fab fragment, a Fab′ fragment, a Fab′-SH fragment, a F(ab′)2 fragment, an Fv fragment, a VHH fragment, a domain antibody, a diabody, and a multispecific antibody or antibody fragment. The eukaryotic cell can be selected from, for example, a mammalian cell, an insect cell, a plant cell, and a fungal cell. The first and second host cells, can, for example, be separately selected from the group consisting of a COS cell, a BHK cell, a HEK293 cell, a DUKX cell, a Saccharomyces spp cell, a Kluyveromyces spp cell, an Aspergillus spp cell, a Neurospora spp cell, a Fusarium spp cell, a Trichoderma spp cell, and a Lepidoptera spp cell. In separate aspects, the first and second host cells are of the same cell type, or of different cell types.

In one aspect, the first and second host cells are grown in the same culture. In another aspect, the first and second host cells are grown in separate cultures. In another aspect, the purifying step may comprise purification using an Obelix cation exchange column. In one aspect, the first host cell does not express a nucleic acid encoding an immunoglobulin light chain, and wherein the second host cell does not express a nucleic acid encoding an immunoglobulin heavy chain. In another aspect, the first and second nucleic acids are derived from one or more monoclonal antibody-producing cells. The monoclonal antibody-producing cells can, for example, be selected from a hybridoma, a polydoma, and an immortilized B-cell.

In one aspect, the refolding comprises mixing the first and second polypeptides under conditions selected from: (a) a ratio of first to second polypeptide of about 1:1, a temperature of about room temperature, and a pH of about 7; and (b) a ratio of first to second polypeptide of about 1:1, a temperature of about 5° C., and a pH in the range of about 8.0 to 8.5. In one further aspect, the first and second polypeptides are mixed in a solution comprising about 0.5 M L-arginine-HCl, about 0.9 mM oxidized glutathione (GSSG), and about 2 mM EDTA.

The invention also provides a method of producing an immunoglobulin molecule or an immunologically functional fragment thereof, the method comprising:

(a) transforming a first host cell with a first nucleic acid comprising a nucleotide sequence encoding a first polypeptide comprising at least the variable domain of an immunoglobulin heavy chain;

(b) transforming a second host cell with a second nucleic acid comprising a nucleotide sequence encoding a second polypeptide comprising at least the variable domain of an immunoglobulin light chain;

(c) expressing the first and second nucleic acid sequences;

(d) dialysing a solution comprising a mixture of the first and second polypeptides; and

(e) allowing the first and second polypeptides to refold to form an immunoglobulin molecule or immunologically functional immunoglobulin fragment;

wherein the first and second host cells are separately selected from the group consisting of a eukaryotic cell and a Gram-positive bacterium. In one aspect, the first and second host cells are grown in the same culture, and the solution is the culture medium in which the first and second host cells are grown. In another aspect, the relative amount of the first and second polypeptides in the solution is in the range of about 1:2 to about 2:1.

The invention also provides for a method of purifying antibodies, the method comprising applying a solution comprising antibodies on an Obelix cation exchange column, and eluting purified antibodies. In one aspect, the method comprises at least one of the following steps: (a) applying filtrated cell culture on the column, the filtrated cell culture optionally being pH adjusted; (b) adding a solvent to the eluation buffer; and (c) eluting antibodies by increasing the salt gradient. In a particular aspect, step (c) is performed before step (b). Alternative elution strategies include, but are not limited to, the use of an elution buffer having a pH of about 6.0 and containing a salt and glycerol (e.g., about 30 mM Citrate, about 25 mM NaCl, about 30% Glycerol at a pH of about 6.0), an elution buffer having a pH of about 7.5-8.5 (e.g., Tris-buffer), a pH gradient from about pH 6.0 to a pH in the range of about 6 to about 9 (e.g., pH 7.5-8.5), and a gradient elution with salt (e.g., NaCl) from 0 to about 1 M at a pH of about 6.5 to about 7.0.

The invention also provides a method for producing an immunoglobulin molecule or an immunologically functional immunoglobulin fragment, comprising at least the variable domains of the immunoglobulin heavy and light chains, said method comprising the steps of:

(a) independently producing the heavy and the light chains in two separate host cells chosen from the group consisting of eukaryotic cells and gram positive bacteria;

(b) purifying the heavy and light chains; and

(c) refolding the immunoglobulin molecule or an immunologically functional immunoglobulin fragment in vitro.

These and other aspects will be more fully described below.

### BRIEF DESCRIPTION OF THE DRAWINGS

FIG. 1 shows a schematic illustration of expression of antibody heavy and light chains, and subsequent ex vivo assembly into full length functional antibodies.

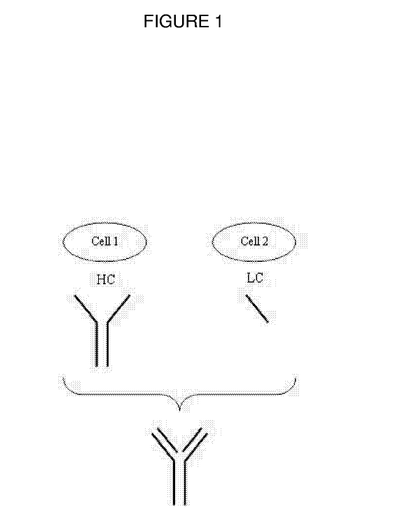

FIG. 2 shows HPLC chromatography of hGH3 antibody after denaturation and reduction as described in Example 1.

FIG. 3 shows SDS-PAGE and Coomassie stain of denatured and reduced hGH3 antibody (as described in Example 1) prior to HPLC purification (lane 2) and HPLC fraction 5 (lane 3), fraction 6 (lane 4), fraction 7 (lane 5), fraction 8 (lane 6). Lane 1 represents a Mark12 marker.

FIG. 4 shows SDS-PAGE analysis of a Mark12 marker (Lane 1) and a sample after denaturation, refolding, and size-exclusion chromatography of hGH3 antibody (Lane 2).

FIG. 5 shows a chromatogram of IgG purification by Obelix cation exchanger.

FIG. 6 shows a non-reduced SDS-PAGE analysis of fractions obtained from purification of IgG on Obelix cation exchanger. Lane 1: Mark12 marker; Lane 2: IgG standard; Lane 3: Application; Lane 4: Run through; Lane 5: Fraction C7; Lane 6: Fraction D7; Lane 7: Fraction E1; Lane 8: Fraction E2, Lane 9: Fraction E3; Lane 10: Fraction E4.

### DESCRIPTION OF THE INVENTION

The present invention is based on the discovery that the separate polypeptide chains of an immunoglobulin molecule, or fragments thereof, can be independently produced in separate host cells, and thereafter refolded to form the final molecule. The host cells can either be co-cultured in the same medium, or cultured separately. After expression, both intra- and interchain disulfide formation is carried out ex vivo under suitable reaction conditions, allowing for the formation of correctly assembled and folded antibodies.

The host cells comprising and encoding immunoglobulin heavy (HC) and light (LC) chains (or fragments of the same), respectively, can be of the same, identical cell type, e.g. from the same mammalian cell line. Alternatively, the host cells respectively expressing HC and LC can be of different origin, e.g., the HC being expressed in a mammalian host cell, and the LC being expressed in a gram-positive bacterium. Furthermore, the DNA sequences encoding the heavy and the light chains may be obtained from one hybridoma cell line, or from more than one monoclonal antibody-producing hybridoma.

As mentioned above, the host cells expressing the heavy and light chains, respectively, can be grown in separate cultures and individual media, or co-cultured in the same media. After recombinant production in separate host cells, the HC and LC chains can be purified by methods known in the art or by methods described herein. For example, the examples describe purification using a highly hydrophobic resin that can bind protein with high conductivity (Obelix cation exchange column, commercially available from Amersham, catalog No. 11-0010).

The final step remaining in order to provide a complete immunoglobulin molecule or a functional immunoglobulin fragment involves the reassembly of the heavy and light chains by disulfide bond formation which in the present invention is referred to as refolding. Refolding, also termed renaturing, can be performed as described in Jin-Lian Xing et al. (2004; World J Gastroenterol 10(14):2029-2033) and Lee and Kwak (2003; Journal of Biotechnology 101:189-198). In a particular embodiment, refolding is achieved by dialysis of a mixture of heavy and light chains (or fragments thereof), the amount of heavy chain and light chain in the mixture being in the range from 1:2 to 2:1. In a further embodiment the range is about 1:1. In the embodiment where the host cells are contained in the same culture medium, the HC and LC (or fragments thereof) self-assemble in the medium, and functional immunoglobulins or fragments can be harvested from the medium. A dialysis step of the culture media containing the mixture of HC and LC can optionally be included in the refolding process.

In one embodiment, the host cell that expresses the HC chain does not contain or express DNA encoding a LC chain, and/or the host cell that expresses the LC chain does not contain or express DNA encoding a HC chain.

The method of the invention provides several advantages compared to the expression of immunoglobulin in a gram negative bacteria, such as E. coli. The advantages include:

(i) no endotoxins are present,

(ii) higher yield of protein is obtained, since there is no need for refolding protein from inclusion bodies,

(iii) full length immunoglobulins can be generated, and

(iv) the glycosylation pattern of the antibody can be modulated depending on the host organism.

Regarding item (i), endotoxins as used herein means toxic activities of enterobacterial lipopolysaccharides and are found in the outer membrane of gram-negative bacteria.

Regarding items (ii) and (v), gram negative bacteria, such as E. coli, are not well suited as production host cells if large quantities of protein are desired. The result of producing large quantities of a desired protein in E. coli is often the formation of inclusion bodies and subsequent refolding. By contrast, gram-positive bacteria have no outer membrane but a glycan layer through which proteins are secreted directly from the cytoplasm into the extracellular space. The relative simple export mechanism facilitates secretion of recombinant proteins in high yields.

Regarding item (iii), due to the large size of full length immunoglobulin molecules, these are difficult to obtain in E. coli. For a recent report on refolding complete IgG molecules produced in E. coli see Simmons et al 2002 J. Immunol. Methods 263:133-147.

Regarding item (iv), most proteins developed for pharmaceutical applications have oligosaccharides attached to their polypeptide backbone, when produced in a eukaryotic host cell. In general sugar chains of such glycoproteins may be attached by N-glycosidic bonds to the amide group of asparagine residues or O-glycosidic bonds to the hydroxyl group of serine or threonine residues. Glycosylation is often required for proper function of the protein and ensures proper folding, function and stability. Prokaryotic organisms lack the ability to perform posttranslational modifications of proteins and glycosylation of proteins is therefore not obtained such systems. Fungi and yeast cells can be engineered to produce proteins with suitable glycosylation patterns (Ballew and Gerngross 2004 Expert Opin. Biol. Ther. 4:623-626).

According to the present invention the above mentioned advantages are provided by independently producing the heavy and the light chains in two separate host cells chosen from the group consisting of eukaryotic cells, and gram positive bacteria. In this context, the term “independently” means that the production of the respective heavy chain (HC) and light chain (LC) (or fragments thereof) can be independently controlled or regulated by use of, e.g., different host cells, different culture media, different expression vectors, and/or different physical conditions (e.g., temperature, redox conditions, pH) of host cell culture. After production of the HC and LC chains (or fragments thereof), ex vivo refolding into a full-length antibody or antibody fragment can be achieved directly in the culture media (if the two separate host cells expressing the HC and LC chain, respectively, are in the same cell culture), or after one or more of joint or separate purification steps of the LC and HC or fragments as described elsewhere herein, dialysis to concentrate the HC and/or LC chain solutions and/or to change buffer, and transfer into or dilution with a particular refolding buffer. Refolding conditions can be selected or optimized for each antibody or antibody fragment according to known methods in the art. Typically, refolding can be obtained at temperatures ranging from about +4° C. to about +40° C., or from about +4° C. to about room temperature, and at a pH ranging from about 5 to about 9, or from about 5.5 to about 8.5. Exemplary buffers that may be used for optimizing refolding include phosphate, citrate-phosphate, acetate, and Tris, as well as cell culture media with pH-regulation by CO₂. Particular refolding conditions are described in Example 1. Other exemplary refolding conditions include a HC:LC (or HC:LC fragment) ratio of about 1:1, a temperature of about room temperature, and a neutral pH. Another exemplary refolding condition include a HC:LC (or HC:LC fragment) ratio of about 1:1, a temperature at about 5° C., about 0.1 M Tris-HCl buffer, about 0.5 M L-arginine-HCl, about 0.9 mM oxidized glutathione (GSSG) as redox system and about 2 mM EDTA at pH of about 8.0-8.5. In one aspect, the refolding solution is dialysed against 20 mM Tris-HCl buffer having a pH of about 7.4, and comprising about 100 mM urea until the conductivity in the equilibrated dialysis buffer has been reduced to a value in the range of about 3.0 to 3.5 mS.

### Antibodies

The terms “antibody” and “immunoglobulin molecule” are used interchangeably herein, and refer to monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM, commonly used in physiological/clinical situations and easily made in a laboratory setting, are exemplary classes of antibodies for employment in this invention.

The immunoglobulin of “IgG” molecules according to the invention may be complete antibodies or they may be functional immunoglobulin fragments. Methods of making fragments or derivatives of the monoclonal antibody are disclosed below.

The skilled person will know how to provide a desired immunoglobulin molecule, and further details are also given below. The immunoglobulin molecule may be, for example, an IgA, IgD, IgE, IgG, or IgM immunoglobulin.

As used herein, a “heavy chain (HC)” or “light chain (LC)” comprises the heavy and light chain of a full-length antibody or a heavy chain and light chain of an antibody fragment.

As used herein, an “antibody fragment” or “immunoglobulin molecule fragment” comprises a portion of a full-length antibody, and is capable of binding an antigen. Typically, an antibody fragment comprises at least one, two, three, four, five, or all CDR-regions of an antibody, or the entire variable heavy (VH) or variable light (VL) portions of the HC and LC, respectively. Exemplary antibody fragments include, but are not limited to, Fab, F(ab)₂, F(ab′)2, Fd, scFv, dsFv fragments, VHH fragments, domain antibodies (VH and/or VL), as well as multispecific (including bi-specific) antibody constructs comprising antigen-binding portions of two or more full-length antibodies.

The term “immunogen” is a substance that is able to induce a humoral antibody and/or cell-mediated immune response rather than immunological tolerance. The term ‘immunogen’ is sometimes used interchangeably with ‘antigen’, yet the term specifies the ability to stimulate an immune response as well as to react with the products of it, e.g. antibody. By contrast, ‘antigen’ is reserved by some to mean a substance that reacts with antibody. The principal immunogens are proteins and polysaccharides, free or attached to microorganisms.

The term “ex vivo” as used herein means outside of any body (i.e., the process does not take place in vivo) and outside of any living cell (i.e., the process does not take place intracellularly).

The term “immunogenic” means the capacity to induce humoral antibody and/or cell-mediated immune responsiveness.

### Antibody Production

The present invention relates to the recombinant production of immunoglobulin molecules or fragments in which at least the variable domains of the heavy-(H) and light-(L) chains are produced in separate host cells, optionally isolated, and refolded in vitro. The VH and VL sequences for application in this invention can be obtained from antibodies produced by any one of a variety of techniques known in the art. Typically, they are provided by immunization of a non-human animal, preferably a mouse, with an immunogen comprising a desired antigen or immunogen. Alternatively, antibodies may be provided by selection of combinatorial libraries of immunoglobulins, as disclosed for instance in Ward et al (Nature 341 (1989) 544).

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988)). In a preferred embodiment, the non-human animal is a mammal, such as a rodent (e.g., mouse, rat, etc.), bovine, porcine, horse, rabbit, goat, sheep, etc. Also, the non-human mammal may be genetically modified or engineered to produce “human” antibodies, such as the Xenomouse™ (Abgenix) or HuMAb-Mouse™ (Medarex). Typically, the immunogen is suspended or dissolved in a buffer, optionally with an adjuvant, such as complete Freund's adjuvant. Methods for determining the amount of immunogen, types of buffers and amounts of adjuvant are well known to those of skill in the art and are not limiting in any way on the present invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with adjuvant such as incomplete Freund's adjuvant. The recall injections, are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be utilized as long as they result in the production of B cells expressing an antibody directed to the antigen used in immunization.

In an alternate embodiment, lymphocytes from a non-immunized non-human mammal are isolated, grown in vitro, and then exposed to the immunogen in cell culture. The lymphocytes are then harvested and the fusion step described below is carried out.

For monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and re-suspended in a buffer that lyses any red blood cells. The solution is again centrifuged and remaining lymphocytes in the pellet are finally re-suspended in fresh buffer.

Once isolated and present in single cell suspension, the lymphocytes are fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in (Goding, “Monoclonal Antibodies: Principles and Practice,” pp. 59-103 (Academic Press, 1986)).

The cells are allowed to grow in the selection media for sufficient time for colony formation and antibody production. This is usually between 7 and 14 days. The hybridoma colonies are then assayed for the production of antibodies that bind the immunogen/antigen. The assay is typically a colorimetric ELISA-type assay, although any assay may be employed that can be adapted to the wells that the hybridomas are grown in. Other assays include immunoprecipitation and radioimmunoassay. The wells positive for the desired antibody production are examined to determine if one or more distinct colonies are present. If more than one colony is present, the cells may be re-cloned and grown to ensure that only a single cell has given rise to the colony producing the desired antibody. Positive wells with a single apparent colony are typically recloned and re-assayed to insure only one monoclonal antibody is being detected and produced.

Hybridomas that are confirmed to be producing a monoclonal antibody are then grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Amersham Biosciences, publication No. 18-1037-46, Edition AC, the disclosure of which is hereby incorporated by reference). The bound antibody is typically eluted from protein A or protein G columns by using low pH buffers (glycine or acetate buffers of pH 3.0 or less) with immediate neutralization of antibody-containing fractions. These fractions are pooled, dialyzed, and concentrated as needed.

As a specific aspect of the invention, the Obelix cation exchanger can be used in the purification of antibodies. The Obelix cation exchanger binds antibodies at high conductivity and at higher pH than pl (for an antibody). This influences the purification capability. The purification can be further modulated by adding, for example, propylendiol so that a hydrophobic interaction can be utilised on this cation exchange column.

DNA encoding the monoclonal antibodies to be used in the method of the invention is readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as bacterial cells, simian COS cells, Chinese hamster ovary (CHO) cells, or myeloma cells that do not otherwise produce immunoglobulin protein, to obtain the synthesis of monoclonal antibodies in the recombinant host cells. Recombinant expression in bacteria of DNA encoding an antibody is well known in the art (see, for example, Skerra et al., Curr. Opinion in Immunol., 5, pp. 256 (1993); and Pluckthun, Immunol. Revs., 130, pp. 151 (1992). For example, the DNA encoding an antibody that binds a desired antigen is isolated from the hybridoma, placed in an appropriate expression vector for transfection into an appropriate host. The host is then used for the recombinant production according to the invention of the antibody, or fragments thereof, such as a humanized version of that monoclonal antibody, active fragments of the antibody, or chimeric antibodies comprising the antigen recognition portion of the antibody.

The DNA sequences encoding the immunoglobulin polypeptides are usually inserted into a recombinant vector which may be any vector, which may conveniently be subjected to recombinant DNA procedures, and the choice of vector will often depend on the host cell into which it is to be introduced. The vector components generally include, but are not limited to, one or more of the following: a promoter, a signal sequence, an origin of replication, one or more selection markers, and a transcription terminator sequence. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the immunoglobulin polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

Expression vectors for use in expressing polypeptides will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell.

Examples of suitable promoter for directing the transcription of the cDNA encoding polypeptide variant in Gram-positive bacteria are the AmyP (Krüger et al 2002 Nature Biotechnol 20:702-706), NisA (de Ruyter et al 1996 J. Bacteriol 178:3434-3439), Spac (Yansura and Henner 1984 Proc. Natl. Acad. Sci. USA 81:439-443), XylA (Eichenbaum et al 1998 Appl. Environ. Microbiol. 63:3451-3457), LacA (Platteeuw et al 1994 Appl. Environ. Microbiol. 60:587-593), UW85 upp (Dunn and Handelsman 1999 Gene 226:297-305).

Examples of suitable promoters for directing the transcription of the DNA encoding the polypeptide variant in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral alpha-amylase, A. niger acid stable alpha-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the human polypeptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. Expression vectors may also contain a set of RNA splice sites located downstream from the promoter and upstream from the insertion site for the polypeptide sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 EIb region, the human growth hormone gene terminator (DeNoto et al. Nucl. Acids Res. 9:3719-3730, 1981) or the polyadenylation signal from the gene. The expression vectors may also include a noncoding viral leader sequence, such as the adenovirus 2 tripartite leader, located between the promoter and the RNA splice sites; and enhancer sequences, such as the SV40 enhancer.

To direct the polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the polypeptides in the correct reading frame. Secretory signal sequences are commonly positioned 5′ to the DNA sequence encoding the peptide. The secretory signal sequence may be that, normally associated with the protein or may be from a gene encoding another secreted protein. In Gram-positive bacteria the signal sequence of the α-amylase gene of L. amylovorus has been used (Krüger et al 2002 Nature Biotechnol 20:702-706)

For secretion from yeast cells, the secretory signal sequence may encode any signal peptide, which ensures efficient direction of the expressed polypeptides into the secretory pathway of the cell. The signal peptide may be naturally occurring signal peptide, or a functional part thereof, or it may be a synthetic peptide. Suitable signal peptides have been found to be the alpha-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the polypeptides. The function of the leader peptide is to allow the expressed peptide to be directed from the endoplasmic reticulum to the Golgi apparatus and further to a secretory vesicle for secretion into the culture medium (i.e. exportation of the polypeptides across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral alpha-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the polypeptides, the promoter and optionally the terminator and/or secretory signal sequence, respectively, and to insert them into suitable vectors containing the information necessary for replication, are well known to persons skilled in the art (see, for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d: 456-467, 1973) or electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker may be an amplifiable selectable marker. A preferred amplifiable selectable marker is a dihydrofolate reductase (DHFR) sequence. Selectable markers are reviewed by Thilly (Mammalian Cell Technology, Butterworth Publishers, Stoneham, Mass., incorporated herein by reference). The person skilled in the art will easily be able to choose suitable selectable markers.

Selectable markers may be introduced into the cell on a separate plasmid at the same time as the gene of interest, or they may be introduced on the same plasmid. On the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the polypeptide variants of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby in-creasing expression levels. Clones of stably transfected cells are then screened for expression of the polypeptide variant of interest.

The host cell into which the DNA sequences encoding the immunoglobulin polypeptides is introduced may be any cell, which is capable of producing the posttranslational modified polypeptides if desired and includes yeast, fungi and higher eukaryotic cells. No posttranslational modifications are obtained in prokaryotic expression systems.

In one embodiment of the invention eukaryotic cells are selected from mammalian cells, insect cells, plant cells, and fungal cells (including yeast cells).

Examples of prokaryotic cells can be Gram-negative cells such as E. coli (Cabilly et al U.S. Pat. No. 6,331,415) or Gram-positive bacteria such as Bacilli, Clostridia, Staphylococci, Lactobailli or Lactococci (de Vos et al 1997 Curr. Opin. Biotechnol. 8:547-553). Exemplary methods of expressing recombinant proteins in Gram-positive bacteria are described in U.S. Pat. No. 5,821,088.

Examples of mammalian cell lines for use in the present invention are the COS-1 (ATCC CRL 1650), baby hamster kidney (BHK) and HEK293 (ATCC CRL 1573; Graham et al., J. Gen. Virol. 36:59-72, 1977) cell lines. A preferred BHK cell line is the tk-ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk-ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous poly-peptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (see, Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans and A. niger. The use of Aspergillus spp. for the expression of proteins is described in, e.g., EP 272 277, EP 238 023, EP 184 438 The transformation of F. oxysporum may, for instance, be carried out as described by Malardier et al., 1989 (Gene 78: 147-156). The transformation of Trichoderma spp. may be performed, for instance, as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the immunoglobulin polypeptides after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The polypeptides produced by the cells may then be recovered or purified from the culture medium by conventional procedures, including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question. In chromatographic procedures, the polypeptides are eluted from the column in a solution. In one aspect, the polypeptides are dialysed before or after purification from culture media to achieve polypeptides in a desired solution.

### Fragments and Derivatives of a Monoclonal Antibody

Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies (such as bispecific antibodies) formed from antibody fragments. “Immunoreactive fragments” comprise a portion of the intact antibody, generally the antigen binding site or variable region. The immunologically functional immunoglobulin fragments produced according to the present invention can comprise at least the variable domain of a heavy chain (VH chain) and at least the variable domain of a light chain (VL chain) of one or more antibodies.

Two exemplary antigen-binding immunoglobulin fragments for which production the method of the invention can be used are Fab and Fv. The smaller Fv (i.e., fragment variable) is composed of the VL and VH regions only. The recombinant version of the Fv is termed the single-chain variable fragment (ScFv). The two fragments in the ScFv are artificially joined with a flexible peptide linker, usually a 15 amino acid linker is used with the sequence (G4S)₃, and expressed as a single polypeptide chain. The linker allows the association of the VH and VL to form the antigen-binding site. The larger Fab (antigen binding fragment) consists of two polypeptides, one containing the light chain variable and constant domains VL-CL, the other a truncated heavy chain containing the variable domain and one constant domain VH-CH1. Just as in intact IgG immunoglobulins, the two chains are linked together by a disulfide bond. ScFv antibodies contain two disulfide bonds, whereas Fabs contain five disulfides that must all form for stable folding.

Expression of ScFv and Fab fragments has been performed in a variety of host cells including eukaryots, yeasts, fungi and bacteria. In Gram-negative bacteria, the oxidation of cysteine thiols into disulfides normally occurs only after a protein has been exported from the highly reducing environment of the cytoplasm to the more oxidizing environment in the periplasm. Proteins expressed into the periplasm can be recovered by osmotic shock or from total cell lysates. Prolonged high-level expression of antibodies at 37° C. renders the outer membrane of E. coli permeable, and the protein can be recovered from the culture media. However, the presence of lipids in the outer membrane constitutes a problem in regards to endotoxin contamination. Gram-positive bacteria and yeasts have no outer membrane and proteins can be secreted directly from the cytoplasm into the extracellular space. As a consequence, no endotoxins are present, and the relatively simple export mechanism facilitates secretion of recombinant proteins in high yields.

Fragments and derivatives of antibodies of this invention can be produced by techniques that are known in the art. For instance, Fab or F(ab′)₂ fragments may be produced by protease digestion of the isolated antibodies, according to conventional techniques. Alternatively, the DNA of a hybridoma producing an antibody of this invention may be modified so as to encode for a fragment of this invention. The modified DNA is then inserted into an expression vector and used to transform or transfect an appropriate cell, which then expresses the desired fragment.

Other types of fragments include variable domain of heavy chain of heavy-chain antibody (VHH) fragments (described in, e.g., Muyldermans, Reviews in Mol. Biotechnol. 2001, 74:277-302 and Spinelli et al., Biochemistry. 2000; 39(6): 1217-22) and domain antibodies (dAbs), which are the smallest functional binding units of antibodies, corresponding to the variable regions of either the heavy (VH) or light (VL) chains of human antibodies. Domain antibodies and exemplary methods to prepare them are described in, e.g., U.S. Pat. No. 6,696,245, WO05/035572, WO04/101790, WO04/081026, WO04/058821, WO04/003019 and WO03/002609.

Multispecific antibodies or antibody fragments can have any suitable number of specificities, including one, two (bispecific) or three specificities. For example, bispecific antibodies have been produced by a variety of known methods including fusion of hybridomas or linking of Fab′ fragments (see, e.g., Songsivilai & Lachmann Clin. Exp. Immunol. 79: 315-321 (1990) and Kostelny et al. J. Immunol. 148:1547-1553 (1992)), and methods of preparing trispecific antibodies are known in the art (see, e.g., Tuft et al. J. Immunol. 147: 60 (1991)).

In an alternate embodiment, the DNA of a hybridoma producing an antibody of this invention can be modified prior to insertion into an expression vector, for example, by substituting the coding sequence for human heavy- and light-chain constant domains in place of the homologous non-human sequences (e.g., Morrison et al., Proc. Natl. Acad. Sci. U.S.A., 81, pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody of the invention.

According to another embodiment, the antibody of this invention is humanized. “Humanized” forms of antibodies according to this invention are specific chimeric immunoglobulins, immunoglobulin chains or fragments thereof (such as Fv, Fab, Fab′, F(ab′)₂, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody. In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the framework regions (FR regions) are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al., Nature, 332, pp. 323 (1988); and Presta, Curr. Op. Struct. Biol., 2, pp. 593 (1992).

Methods for humanizing the antibodies for use in the invention are well known in the art. Generally, a humanized antibody according to the present invention has one or more amino acid residues introduced into it from the original antibody. These murine or other non-human amino acid residues are often referred to as “import” residues, which are typically taken from an “import” variable domain. Humanization can be essentially performed following the method of Winter and co-workers (Jones et al., Nature, 321, pp. 522 (1986); Riechmann et al., Nature, 332, pp. 323 (1988); Verhoeyen et al., Science, 239, pp. 1534 (1988)). Accordingly, such “humanized” antibodies are chimeric antibodies (U.S. Pat. No. 4,816,567 to Cabilly et al.), wherein substantially less than an intact human variable domain has been substituted by the corresponding sequence from the original antibody. In practice, humanized antibodies according to this invention are typically human antibodies in which some CDR residues and possibly some FR residues are substituted by residues from analogous sites in the original antibody.

The choice of human variable domains, both light and heavy, to be used in making the humanized antibodies is very important to reduce antigenicity. According to the so-called “best-fit” method, the sequence of the variable domain of an antibody of this invention is screened against the entire library of known human variable-domain sequences. The human sequence which is closest to that of the mouse is then accepted as the human framework (FR) for the humanized antibody (Sims et al., J. Immunol., 151, pp. 2296 (1993); Chothia and Lesk, J. Mol. Biol., 196, pp. 901 (1987)). Another method uses a particular framework from the consensus sequence of all human antibodies of a particular subgroup of light or heavy chains. The same framework can be used for several different humanized antibodies (Carter et al., Proc. Natl. Acad. Sci. U.S.A., 89, pp. 4285 (1992); Presta et al., J. Immunol., 51, pp. 1993)).

Another method is to make “human” monoclonal antibodies using a XenoMouse® (Abgenix, Fremont, Calif.) as the mouse used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963. An analogous method can be achieved using a HuMAb-Mouse™ (Medarex).

Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

The antibodies of the present invention may also be derivatized to “chimeric” antibodies (immunoglobulins) in which a portion of the heavy and/or light chain is identical with or homologous to corresponding sequences in the original antibody, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., supra; Morrison et al., Proc. Natl. Acad. Sci. U.S.A., 81, pp. 6851 (1984)).

The following examples further illustrate particular aspects of the invention, without limitation.

### EXAMPLES

### Example 1

### Separation and Refolding (Renaturation) of Antibody Light and Heavy Chain

The human antibody TF36F40 recognizing human tissue factor (TF) and the murine antibody hGH3 recognizing human growth hormone (hGH) were used in the following. Each antibody was denatured using 6M guanidinium chloride and 50 mM sodium phosphate pH 8.4 and disulphide bonds were reduced by addition of dithiothreithol (DTT) to a final concentration of 100 mM and incubated at room temperature for 30 minutes before analyzing the sample by SDS-PAGE and Western blotting under both reduced and unreduced conditions. Peroxidase-conjugated goat-anti-human IgG heavy chain and goat-anti-human kappa light chain were applied for detection of TF36F40 heavy chain (HC) and light chain (LC), respectively. Peroxidase-conjugated goat-anti-murine IgG heavy chain and goat-anti-murine kappa light chain were applied for detection of hGH3 heavy and light chain, respectively.

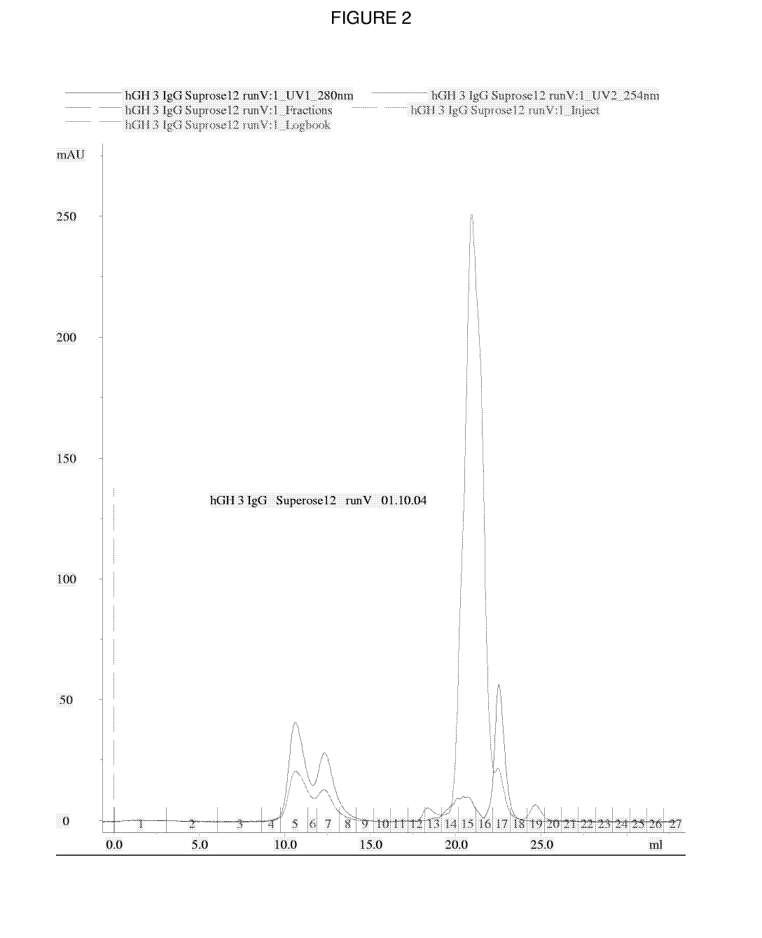

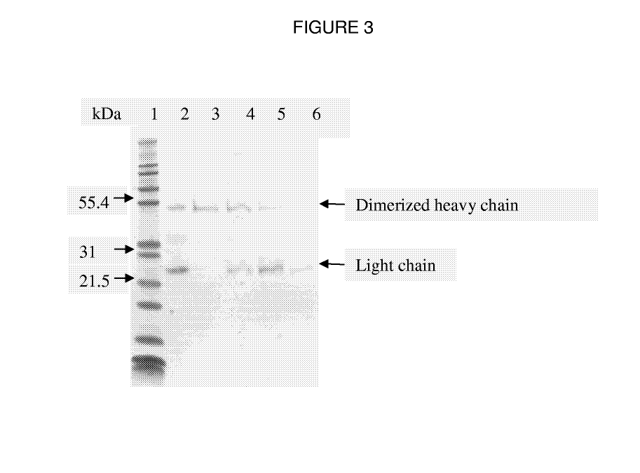

Light and heavy chains of each antibody were subsequently separated by Size Exclusion Chromatography, SEC, using a Superose 12 column (Amersham Bioscience) and the washing and equilibration conditions provided by the manufacturer. The proteins were eluted from the column using 6 M guanidinium chloride, 50 mM sodium phosphate pH 8.4 and 50 mM DTT and the fractions were analyzed by SDS-PAGE and Western blotting as described above (FIGS. 2 and 3).

In order to refold the hGH3 antibody, light and heavy chain fractions were mixed in an equimolar ratio and dialyzed against 100 mM Tris HCL, pH 7.5 and 10 mM EDTA for 1 hour at 37° C. Full length IgG was isolated by size exclusion chromatography in the presence of phosphate-buffered saline (PBS) before passing the refolded antibody through a sterile 0.2 μm filter. The sample was stored at 4° C. until use.

In an alternative experiment, to refold the hGH3 antibody, light and heavy chain fractions were mixed in an equimolar ratio and dialyzed against the following buffers:

1) 4 M Guanidinium hydrochloride, 50 mM Na Phosphate, 5 mM DTT, pH 8.4 for 90 min at room temperature;

2) 2 M Guanidinium hydrochloride, 50 mM Na Phosphate, 5 mM DTT, pH 8.4 for 90 min at room temperature;

3) 1 M Guanidinium hydrochloride, 50 mM Na Phosphate, 5 mM DTT, pH 8.4 for 90 min at room temperature;

4) 0.5 M Guanidinium hydrochloride, 50 mM Na Phosphate, 5 mM DTT, pH 8.4 for 90 min at room temperature; and

5) 50 mM Na Phosphate, 5 mM DTT, pH 8.4 for 72 hrs at room temperature, to allow oxidation of DTT and disulfide bridges.

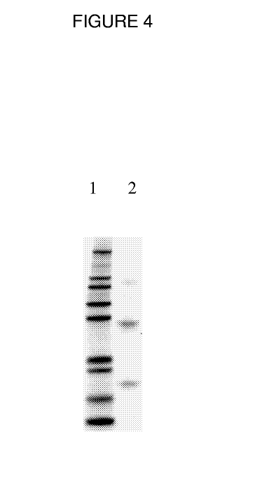

Final volume was 2 ml. 500 ul was concentrated by ultrafiltration in a centricon filter with a cut-off of 14 kDa. Both solutions were subsequently analysed by SDS PAGE (FIG. 4).

### Example 2

### ELISA

The in vitro assembled HuTF36F40 and hGH3 antibodies are assayed by ELISA using standard procedures. Briefly, ELISA microtiter plates (Maxisorp, Nunc, Denmark) are coated overnight at 4° C. with 1 μg/ml human hGH or human TF (TF1-119) in 1×PBS. In vitro assembled hGH3 or HuTF36F40 antibody sample is added and the binding to hGH or human TF is detected using peroxidase-conjugated goat-anti-Human or -murine IgG (Sigma), respectively.

### Example 3

### Surface Plasmon Resonance

A Biacore 3000 optical biosensor is used to evaluate the affinities of the in vitro assembled antibodies towards hGH or human TF. In order to determine affinities approx 10000 RU of antigen is immobilized to the sensor surface by EDC/NHS coupling chemistry. Thereafter, the antibody is injected into the flow cell with a flow rate of about 5 μl/min for about 3 min and allowed to associate with its respective antigen (hGH or human TF). Following the association phase, the surface is washed with running buffer (HBS-EP, pH 7.4, containing 0.005% detergent P20) at a flow rate of 5 μl/min for 2 min. The sensorgram data are analyzed using the Bia evaluation software 3.0.

### Example 4

### Isolation of Total RNA from Hybridoma Cells

4×10⁶ hybridoma cells (TF-36F40) secreting antibodies against tissue factor are used for isolation of total RNA using RNeasy Mini Kit from Qiagen. The cells are pelleted for 5 min at 1000 rpm and disrupted by addition of 350 μl RLT buffer containing 10 μl/ml beta-mercaptoethanol. The lysate is transferred onto a QIAshredder column from Qiagen and centrifuged for 2 min at maximum speed. The flow through is mixed with 1 volume 70% ethanol. Up to 700 μl sample is applied per RNeasy spin column and centrifuged at 14000 rpm and the flow through discarded. 700 μl RW1 buffer is applied per column and centrifuged at 14000 rpm for 15s to wash the column. The column is washed twice with 500 μl RPE buffer and centrifuged for 14000 rpm for 15s. To dry the column it is centrifuged for additionally 2 min at 14000 rpm. The column is transferred to a new collection tube and the RNA is eluted with 50 μl of nuclease-free water and centrifuged for 1 min at 14000 rpm. The RNA concentration is measured by absorbance at OD=260 nm. The RNA is stored at −80° C. until needed.

### Example 5

### cDNA Synthesis

1 μg RNA is used for first-strand cDNA synthesis using SMART RACE cDNA Amplification Kit from Clontech. For preparation of 5′-RACE-Ready cDNA, a reaction mixture containing RNA isolated, as described above, backprimer 5′-CDS primer back, and SMART II A oligo, is prepared and incubated at 72° C. for about 2 min., and subsequently cooled on ice for about 2 min. before adding 1× First-Strand buffer, DTT (20 mM), dNTP (10 mM) and PowerScript Reverse Transcriptase. The reaction mixture is incubated at 42° C. for 1.5 hour and Tricine-EDTA buffer is added and incubated at 72° C. for 7 min. Samples can be stored at −20° C.

### Example 6

### Amplification and Cloning of Human Light (LC) and Human Heavy Chains (HC)

A PCR (Polymerase Chain Reaction) reaction mixture containing 1× Advantage HF 2 PCR buffer, dNTP (10 mM) and 1× Advantage HF 2 polymerase mix is established for separate amplification of variable regions of both VL and VH from cDNA made as above.

For amplification of LC the following primers are
used:
UPM (Universal Primer Mix):

(SEQ ID NO:1)

5′-CTAATACGACTCACTATAGGGCAAGCAGTGGTATCAACGCAGAG
T-3′

(SEQ ID NO:2)

5′-CTAATACGACTCACTATAGGG-3′
P1:

(SEQ ID NO:3)

5′-CGCGGCTAGCACACTCTCCCCTGTTGAAGCTC-3′
For amplification of HC the following primers are
used:
UPM (Universal Primer Mix):

(SEQ ID NO:1)

5′-CTAATACGACTCACTATAGGGCAAGCAGTGGTATCAACGCAGAG
T-3′

(SEQ ID NO:2)

5′-CTAATACGACTCACTATAGGG-3′
P2:

(SEQ ID NO:4)

5′-TCATTTACCCGGGGACAGGGAGA-3′

Three rounds of PCR are conducted. Round 1: PCR is run for 5 cycles at 94° C. for 5s and 72° C. for 3 min. Round 2: PCR is run for 5 cycles at 94° C. for 5s, 70° C. for 10s, and 72° C. for 1 min. Round 3: PCR is run for 28 cycles at 94° C. for 5s, 68° C. for 10s, and 72° C. for 1 min.

The PCR products are analyzed by electrophoresis on a 1% agarose gel and the DNA purified from the gel using QIAEX11 agarose gel extraction kit from Qiagen.

The purified PCR products are introduced into PCR4-TOPO vector using TOPO TA Cloning kit from Invitrogen and used for transformation of TOP10 competent cells.

A suitable amount of colonies are analyzed by colony PCR using Taq polymerase, 1×Taq polymerase buffer, dNTP (10 mM) and the following primers and PCR program:

| | |
|---|---|
| M13forward: | |
| 5′-GTAAAACGACGGCCAG-3′ | (SEQ ID NO:5) |
| M13reverse: | |
| 5′-CAGGAAACAGCTATGAC-3′ | (SEQ ID NO:6) |

PCR Program:
25 cycles are run at 94° C. for 30 s, 55° C. for 30 s, and 72° C. for 1 min.

Plasmid DNA from clones comprising LC and HC inserts, respectively, is extracted and sequenced using primer M13forward and M13reverse listed above. The resultant sequences of the TF-36F40 antibody genes are listed as SEQ ID NOS:7 and 8, respectively, and the encoded light and heavy chain sequences are described in SEQ ID NOS:9 and 10, respectively.

### Example 7

### Introduction of Antibody Genes into Mammalian Expression Vectors

Based on the sequence data (SEQ ID NOS:7 and 8) disclosing the LC and HC sequence of human TF-36F40 respectively, primers are designed for the amplification of the LC and the HC genes, respectively. The antibody regions are formatted by PCR to include a Kozak sequence, leader sequence and unique restriction enzyme sites. This is achieved by designing 5′ PCR primers to introduce a HindIII site, the Kozak sequence and to be homologous to the 5′ end of the leader sequence of the variable light chain region. The 3′ primer is homologous to the 3′ end of the C-terminal of the constant light and heavy chain regions, respectively, and introduces a XbaI site at the 3′-end. The HC region is generated in a similar fashion except that a NotI and a BamHI site are introduced in the 5′ and 3′ end instead of HindIII and XbaI, respectively. The DNA fragments are digested with HindIII+XbaI and NotI+BamHI, respectively, and ligated into a the commercially available eukaryotic expression vector (pcDNA3(+) from Invitrogen) containing the beta-lactamase gene encoding resistance to ampicillin and an E. coli replication origin (pUC). The ligated plasmids are used to transform E. coli. Plasmid DNA is prepared from these ampicillin resistant bacterial populations and used for transfection into mammalian cells.

### Example 8

### Transfection of Mammalian Cells

The cloned DNAs described above are introduced into mammalian cells using Lipofectamine™ 2000 (Cat. No. 11668-019, Invitrogen) according to the manufacturer's recommendations.

### Example 9

### Expression of Antibody LC and HC in Mammalian Cells

Cells are grown as described by the manufacturer's protocol (Cat. No. 11668-019, Invitrogen). Cells are grown 6 days prior to harvesting of supernatant and subsequent purification and ex vivo assembly.

Co-culturing of cells expressing LC and HC, respectively, is performed in the following manner: HEK293 cells are transfected with LC and HC vector constructs, respectively, and grown for 16 hours at 37° C. before combining the two cultures. After 6 days, the supernatant from the mixed culture is analyzed as described in examples 2 and 3. In order to optimize the ex vivo assembly of antibodies parameters such as host cell, expression vectors, culture media and/or different biophysical conditions (e.g. temperature, redox conditions, pH) of host cell culture, is varied. The concentration of LC and HC as well as the LC:HC ratio can be optimized by changing these parameters. In one experiment, the following conditions are used: HEK293 cells transfected with pcDNA3(+) based vector constructs expressing antibody LC and HC, respectively, and grown at 37° C. at 200 rpm in Glutamax-I supplemented Optimum1 media (GIBCO cat. no. 51985-026) containing 25 μg/ml Geneticin (GIBCO cat. No. 10131-019).

### Example 10

### Transformation and Expression of Antibody LC and HC in Gram-Positive Bacteria

Based on the sequence data (SEQ ID NOS:7 and 8) disclosing the LC and HC sequence of human TF-36F40 respectively, primers are designed for the amplification of the LC and the HC genes, respectively. The antibody regions are formatted by PCR to include NotI/BamHI sites for HC and HindIII/XbaI sites for LC, respectively, but without the naturally occurring signal peptide.

The HC and LC DNA fragments are digested, and ligated separately into a modified version of the Gram-positive Streptococcus gordonii SPEX expression vector (Myscofski et al 2000 Protein Expression Purif) in which expression is driven by the P2 promoter. The cloned DNAs described above are introduced into gram-positive bacteria as described previously (Myscofski et a/2000 Protein Expression Purif.) Cells are grown in brain-heart infusion broth (BHI, Difco) overnight at 37° C. prior to harvesting of supernatant and subsequent purification and ex vivo assembly.

Co-culturing of cells expressing LC and HC, respectively, is performed in the same manner as described above.

### Example 11

### Purification of Antibody Fragments

The LC is purified from supernatant using protein L chromatography, and HC is purified by protein A chromatography, according to known procedures in the art. Alternatively, the LC is purified from supernatant using antibodies against VLCL immobilized to a matrix using EDC/NHS coupling chemistry, and/or the HC is purified from supernatant using antibodies against VHCH1-3.

### Example 12

### In Vitro Assembly of Antibody Fragments

In vitro assembly of TF36F40 LC and HC into a full length TF antibody is performed as described in example 1. The antibody is quantified using the BCA protein assay (Pierce, Rockford, Ill.) and assayed as described in examples 2 and 3.

### Example 13

### Purification by Obelix Cation Exchanger

The Obelix cation exchanger is commercially available from Amersham (Cat. No. 11-0010).

For use, the pH of the filtrated cell culture is in the range of about 3.0-9.0 (preferably about pH 7.0). The sample is applied to the column in a range of about 5-40 (preferably about 10) mg/ml packed column material.

Unbound material is washed off the column by applying 1-5 CV (column volumes) of buffer A (buffer A is defined as a buffer with pH in the preferred range, with a sufficiently low conductivity to allow binding of the antibody in question).

Subsequently, the antibody is eluted from the column via increasing the conductivity and thus increasing the electrostatic repulsion. This can either be achieved by a step gradient or a continuous gradient from low to high salt concentration. Preferably, NaCl is chosen as salt, but also other types of salts can be used.

Propandiols can be added to elution buffers and in this mode the cation exchanger can be used as a hydrofob interaction chromatography column. Adding a salt with high salting-out effect, e.g., sodium acetate, can alter the cation exchanger to a hydrophobic interaction chromatography column. Thus, by changing parameters such as salts, pH, conductivity and hydrophobicity, the column can function in two ways or more during the same run. The chromatography procedure is typically run at 4-25° C.

In one experiment, the following conditions were used: The antibody (Ab) application was prepared by adjusting pH in the culture to pH 5.5 and filtered on 0.2 μm filter. EDTA and Benzamidine-HCl were added to 1 mM.

The column material was equilibrated with 30 mM Citrate buffer pH 6.0 and the application was loaded at 20 cv/h at room temperature and about 5 mg Ab per ml column material.

After application the column was washed with equilibration buffer. A wash with 30 mM Citrate 25 mM NaCl 30% Glycerol pH 6.0 was performed followed a wash with equilibration buffer.

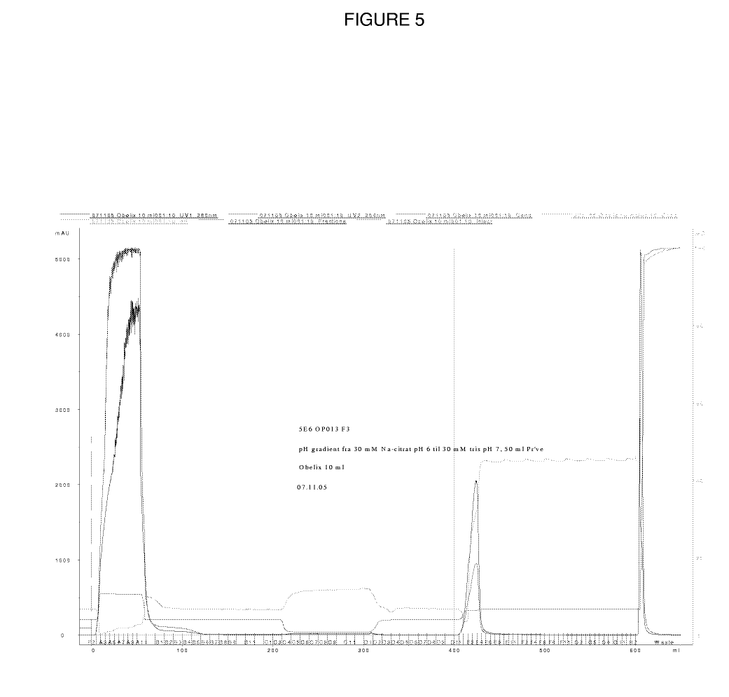

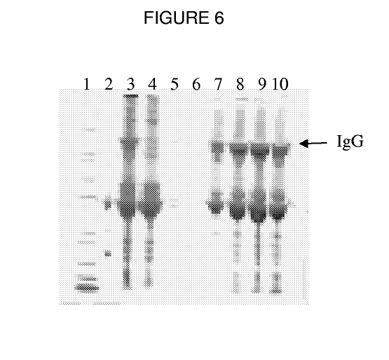

Elution can be done in different ways. In one experiment, elution was performed with Tris-buffer pH 7.5-8.5 (FIGS. 5 and 6). In another experiment, a gradient elution with salt from 0 to 1 M NaCl at pH 6.5-7 was used.

After elution of Ab the column is regenerated with 1 M NaOH.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way,

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

1. A method of producing an immunoglobulin molecule or an immunologically functional fragment thereof, the method comprising:
(a) transforming a first host cell with a first nucleic acid comprising a nucleotide sequence encoding a first polypeptide comprising at least the variable domain of an immunoglobulin heavy chain;
(b) transforming a second host cell with a second nucleic acid comprising a nucleotide sequence encoding a second polypeptide comprising at least the variable domain of an immunoglobulin light chain;
(c) expressing the first and second nucleic acid sequences;
(d) purifying the first and second polypeptides; and
(e) allowing the first and second polypeptides to refold to form an immunoglobulin molecule or immunologically functional immunoglobulin fragment;
wherein the first and second host cells are separately selected from the group consisting of a eukaryotic cell and a Gram-positive bacterium.

2. The method of claim 1, wherein the first host cell does not express a nucleic acid encoding an immunoglobulin light chain, and wherein the second host cell does not express a nucleic acid encoding an immunoglobulin heavy chain.

3. The method of any claim 1, wherein the immunoglobulin molecule is selected from the group consisting of an IgA, an IgD, an IgE, an IgG, and an IgM immunoglobulin.

4. The method of any claim 1, wherein the immunologically functional fragment is selected from the group consisting of a Fab fragment, a Fab′ fragment, a Fab′-SH fragment, a F(ab′)₂ fragment, an Fv fragment, a VHH fragment, a domain antibody, a diabody, and a multispecific antibody or antibody fragment.

5. The method of claim 1, wherein the first and second host cells are grown in the same culture.

6. The method of claim 1, wherein the first and second host cells are grown in separate cultures.

7. The method of claim 1, wherein the purifying comprises purification using an Obelix cation exchange column.

8. The method of claim 1, wherein the eukaryotic cell is selected from the group consisting of a mammalian cell, an insect cell, a plant cell, and a fungal cell.

9. The method of claim 1, wherein the first and second host cells are separately selected from the group consisting of a COS cell, a BHK cell, a HEK293 cell, a DUKX cell, a Saccharomyces spp cell, a Kluyveromyces spp cell, an Aspergillus spp cell, a Neurospora spp cell, a Fusarium spp cell, a Trichoderma spp cell, and a Lepidoptera spp cell.

10. The method of claim 1, wherein the first and second host cells are of the same cell type.

11. The method of claim 1, wherein the first and second host cells are of different cell types.

12. The method of claim 1, wherein the first and second nucleic acids are derived from one or more monoclonal antibody-producing cells.

13. The method of claim 12, wherein the monoclonal antibody-producing cells are selected from the group consisting of a hybridoma, a polydoma, and an immortalized B-cell.

14. The method of claim 7, wherein step (e) comprises mixing the first and second polypeptides under conditions selected from:
(a) a ratio of first to second polypeptide of about 1:1, a temperature of about room temperature, and a pH of about 7; and
(b) a ratio of first to second polypeptide of about 1:1, a temperature of about 5° C., and a pH in the range of about 8.0 to 8.5.

15. The method of claim 14, wherein the first and second polypeptides are mixed in a solution comprising about 0.5 M L-arginine-HCl, about 0.9 mM oxidized glutathione (GSSG), and about 2 mM EDTA.

16. A method of producing an immunoglobulin molecule or an immunologically functional fragment thereof, the method comprising:
(a) transforming a first host cell with a first nucleic acid comprising a nucleotide sequence encoding a first polypeptide comprising at least the variable domain of an immunoglobulin heavy chain;
(b) transforming a second host cell with a second nucleic acid comprising a nucleotide sequence encoding a second polypeptide comprising at least the variable domain of an immunoglobulin light chain;
(c) expressing the first and second nucleic acid sequences;
(d) dialysing a solution comprising a mixture of the first and second polypeptides; and
(e) allowing the first and second polypeptides to refold to form an immunoglobulin molecule or immunologically functional immunoglobulin fragment;
wherein the first and second host cells are separately selected from the group consisting of a eukaryotic cell and a Gram-positive bacterium.

17. The method of claim 16, wherein the first and second host cells are grown in the same culture, and the solution is the culture medium in which the first and second host cells are grown.

18. The method of claim 17, wherein the relative amount of the first and second polypeptides in the solution is in the range of about 1:2 to about 2:1.

19. A method of purifying antibodies, the method comprising applying a solution comprising antibodies on an Obelix cation exchange column, and eluting purified antibodies.

20. The method of claim 19, comprising at least one of the following steps:
(a) applying filtrated cell culture on the column, the filtrated cell culture optionally being pH adjusted;
(b) adding a solvent to the eluation buffer; and
(c) eluting antibodies by increasing the salt gradient.

21. The method of claim 20, wherein the step (c) is performed before step (b).

22. A method for producing an immunoglobulin molecule or an immunologically functional immunoglobulin fragment, comprising at least the variable domains of the immunoglobulin heavy and light chains, said method comprising the steps of:
(a) independently producing the heavy and the light chains in two separate host cells chosen from the group consisting of eukaryotic cells and gram positive bacteria;
(b) purifying the heavy and light chains; and
(c) refolding the immunoglobulin molecule or an immunologically functional immunoglobulin fragment in vitro.
